(19) United States
(12) Patent Application Publication
(10) Pub. No.: US 2023/0009461 A1
(43) Pub. Date: Jan. 12, 2023

(54) MEDICAL DEVICE

(71) Applicant: Terumo Kabushiki Kaisha, Tokyo (JP)

(72) Inventor: Yusuke TAKAHASHI, Hadano-shi (JP)

(73) Assignee: Terumo Kabushiki Kaisha, Tokyo (JP)

(21) Appl. No.: 17/934,357

(22) Filed: Sep. 22, 2022

Related U.S. Application Data
(63) Continuation of application No. PCT/JP2021/012381, filed on Mar. 24, 2021.

(30) Foreign Application Priority Data
Mar. 27, 2020 (JP) 2020-058892

(51) Int. Cl. A61B 90/00

(52) U.S. Cl. CPC A61B 90/02

(57) ABSTRACT

A medical device capable of preventing a circumferential twist of an expansion body expandable in a radial direction and effectively pressing an energy transfer element against biological tissue. The medical device includes an outer tube, an expansion body expandable in a radial direction by contracting along an axis, a pulling shaft, and a plurality of energy transfer elements disposed in the expansion body. The expansion body includes a plurality of main struts in which the energy transfer elements are disposed, and distal side support struts and proximal side support struts which are connected to the main struts. A portion of each of the main struts between a force reception portion receiving a pulling force from the pulling shaft and the energy transfer element is substantially parallel to the axis when viewed from a radially outer side.

### CROSS-REFERENCES TO RELATED APPLICATIONS

This application is a continuation of International Application No. PCT/JP2021/012381 filed on Mar. 24, 2021, which claims priority to Japanese Application No. 2020-058892 filed on Mar. 27, 2020, the entire content of both of which is incorporated herein by reference.

### TECHNOLOGICAL FIELD

The present disclosure generally relates to a medical device that applies energy to biological tissue.

### BACKGROUND DISCUSSION

In recent years, a device has been used, which is inserted into a lumen of a living body such as a blood vessel to enlarge the lumen or hole of the living body. For example, Japanese Patent Application Publication No. 2003-210590 A discloses a catheter including a basket-shaped electrode assembly for mapping electrical activity of the heart. A proximal portion of the electrode assembly is fixed to a distal portion of an outer tube, and a distal portion of the electrode assembly is fixed to a distal portion of an inner tube penetrating the outer tube. The electrode assembly includes a plurality of wires extending along an axis of the inner tube and curved so as to protrude outward in a radial direction, and electrodes disposed on the respective wires. The plurality of wires are substantially parallel to an axis of the electrode assembly when viewed from a radially outer side of the electrode assembly. By pulling the inner tube, the wires of the electrode assembly are compressed in an axial direction and are bent, and protrude outward in the radial direction. Accordingly, the electrodes disposed on the wires are pressed against biological tissue.

When the wires substantially parallel to the axis as viewed from the radially outer side are compressed in the axial direction of the electrode assembly, the wires are likely to be twisted in a circumferential direction around the axis of the electrode assembly. Accordingly, a force for compressing the wires is dispersed, and it is difficult to effectively transmit the force to the tissue.

### SUMMARY

A medical device is disclosed that is capable of preventing a circumferential twist of an expansion body expandable in a radial direction and effectively pressing an energy transfer element against biological tissue.

A medical device is disclosed that includes: an elongated outer tube; an expansion body connected to a distal portion of the outer tube and configured to expand in a radial direction by contracting along an axis of the outer tube; a pulling shaft configured to slide with respect to the outer tube, the pulling shaft being disposed inside the outer tube, protruding from the distal portion of the outer tube, and being connected to a distal portion of the expansion body; and a plurality of energy transfer elements disposed in the expansion body and configured to output energy. The expansion body includes a plurality of main struts arranged at intervals in a circumferential direction and extending along the axis of the outer tube by a predetermined length, and a plurality of sub-struts connected to the plurality of main struts. At least one of the plurality of energy transfer elements is disposed on each of the plurality of main struts. Each of the plurality of main struts includes a force reception portion configured to receive a pulling force from the pulling shaft. A portion of each of the plurality of main struts between the force reception portion and the energy transfer element is substantially parallel to the axis when viewed from a radially outer side. Each of the plurality of sub-struts includes at least one support strut having two joint portions joined respectively to two circumferentially adjacent main struts among the plurality of main struts. Each of the plurality of support struts is formed to be longer than a linear distance between the two joint portions.

In the medical device configured as described above, the support strut prevents the main strut receiving the pulling force from being twisted in the circumferential direction when the energy transfer element is pressed against tissue. Therefore, in the medical device, a force for pressing the energy transfer element against the tissue is less likely to be dispersed, and the energy transfer element can be effectively pressed against the biological tissue.

Each of the plurality of support struts may include two inclined struts respectively extending from two circumferentially adjacent main struts so as to be inclined with respect to the axis when viewed from the radially outer side, and a merging portion connecting the two inclined struts to each other, and the two inclined struts connected to the merging portion may be plane-symmetrical with respect to a plane passing through the merging portion and the axis of the expansion body. Accordingly, when the expansion body is deformed, the two inclined struts that are plane-symmetrical are deformed into a symmetrical shape. Therefore, forces acting on the two circumferentially adjacent main struts from the inclined struts are equal to each other. Therefore, the main strut can be prevented from being twisted in the circumferential direction.

The support struts may be disposed at a plurality of positions in an axial direction of the expansion body. Accordingly, in the medical device, when the energy transfer element is pressed against the tissue, a twist of the main strut in the circumferential direction can be effectively prevented by the plurality of support struts in the axial direction.

The plurality of support struts disposed at the plurality of positions in the axial direction of the expansion body may be connected to each other. Accordingly, in the medical device, when the energy transfer element is pressed against the tissue, the twist of the main strut in the circumferential direction can be effectively prevented by the plurality of support struts connected adjacently in the axial direction. Since the support struts disposed at the plurality of positions in the axial direction are connected to each other, the main strut can be prevented from being bent. Therefore, in the medical device, the force for pressing the energy transfer element against the tissue is less likely to be dispersed, and the energy transfer element can be effectively pressed against the tissue.

The expansion body may include a distal side sandwiching strut and a proximal side sandwiching strut whose separation distance is narrowed by expansion of the expansion body, an inward protruding portion protruding inward in the radial direction may be formed between the distal side sandwiching strut and the proximal side sandwiching strut, and the support strut may be disposed on at least one of a distal side and a proximal side of the inward protruding portion. Accordingly, the distal side sandwiching strut and the proximal side sandwiching strut are less likely to be twisted in the circumferential direction due to the support strut. Therefore, a force for holding the biological tissue by the distal side sandwiching strut and the proximal side sandwiching strut is less likely to be dispersed, and the tissue can be effectively held.

An expansion body is disclosed that is connected to a distal portion of an outer tube and configured to expand in a radial direction, the expansion body comprising: a plurality of main struts arranged at intervals in a circumferential direction and extending along an axis of the expansion body, each of the main struts includes a proximal side main strut, a proximal side sandwiching strut, a distal side sandwiching strut, and a distal side main strut; each of the proximal side main struts being inclined so as to increase in a radial direction from a distal portion of the outer tube toward a distal direction, and the distal side main strut is inclined so as to increase in the radial direction from a force reception portion toward a proximal direction of the expansion body; each of the proximal side sandwiching struts being inclined so as to decrease in the radial direction from a distal portion of the proximal side main strut toward the distal direction; each of the distal side sandwiching struts being inclined so as to decrease in the radial direction from a proximal portion of the distal side main strut toward the proximal direction; wherein the proximal side sandwiching strut and the distal side sandwiching strut are connected to each other by an inward protruding portion protruding inward in the radial direction; and each of the plurality of main struts including an energy transfer element disposed at a position where one of the proximal side sandwiching strut and the distal side sandwiching strut of the main strut such that the energy transfer element sandwiches biological tissue with an other of the proximal side sandwiching strut and the distal side sandwiching strut of the main strut.

A treatment method is disclosed comprising: performing maintenance treatment for maintaining a size of a through-hole formed in an atrial septum to allow a right atrium and a left atrium of a heart failure patient to communicate with each other with a medical device, the medical device including an elongated outer tube, an expansion body connected to a distal portion of the outer tube and configured to expand in a radial direction, a pulling shaft configured to slide with respect to the outer tube, the pulling shaft being disposed inside the outer tube, protruding from the distal portion of the outer tube, and being connected to a distal portion of the expansion body, a plurality of energy transfer elements disposed in the expansion body and configured to output energy, the expansion body including a plurality of main struts arranged at intervals in a circumferential direction and extending along an axis of the outer tube by a predetermined length, and a plurality of sub-struts connected to the plurality of main struts, at least one of the plurality of energy transfer elements is disposed on each of the plurality of main struts, each of the plurality of main struts includes a force reception portion configured to receive a pulling force from the pulling shaft, a portion of each of the plurality of main struts between the force reception portion and the energy transfer element is substantially parallel to an axis of the expansion body when viewed from a radially outer side, each of the plurality of sub-struts includes at least one support strut having two joint portions joined respectively to two circumferentially adjacent main struts among the plurality of main struts, and each of the plurality of support struts is formed to be longer than a linear distance between the two joint portions.

### BRIEF DESCRIPTION OF THE DRAWINGS

FIG. 1 is a plan view showing an overall configuration of a medical device according to an embodiment.

FIG. 2 is a plan view showing a distal portion of the medical device.

FIG. 3 is a front view showing the distal portion of the medical device.

FIG. 4 is a plan view of the distal portion of the medical device with an expansion body shown in a transparent manner.

FIG. 5 is a plan view of the distal portion of the medical device with a pulling shaft shown in a transparent manner.

FIG. 6 is a plan view showing a state in which the expansion body expands using the pulling shaft.

FIG. 7 is an explanatory view schematically showing a state in which the expansion body is disposed in a through-hole of an atrial septum, the medical device being in a plan view, and biological tissue being in a cross-sectional view.

FIG. 8 is an explanatory view schematically showing a state in which the distal portion of the medical device is inserted into the atrial septum, a part of the medical device being in a plan view, and a storage sheath and the biological tissue being in a cross-sectional view.

FIG. 9 is an explanatory view schematically showing a state in which the expansion body is deployed and disposed in the atrial septum, the medical device being in a plan view, and the biological tissue being in a cross-sectional view.

FIG. 10 is an explanatory view schematically showing a state in which a balloon is inflated, the medical device being in a plan view, and the biological tissue being in a cross-sectional view.

FIG. 11 is an explanatory view schematically showing a state in which the expansion body expands, the medical device being in a plan view, and the biological tissue being in a cross-sectional view.

FIG. 12 is a plan view showing a first modification of the medical device.

FIG. 13 is a plan view showing a second modification of the medical device.

FIG. 14 is a plan view showing a third modification of the medical device.

FIG. 15 is a plan view showing a fourth modification of the medical device.

FIG. 16 is a plan view showing a fifth modification of the medical device.

FIG. 17 is a plan view showing a sixth modification of the medical device.

FIGS. 18A and 18B are plan views showing a seventh modification of the medical device, in which FIG. 18A shows a deployed form and FIG. 18B shows an expansion form.

FIG. 19 is a plan view showing an eighth modification of the medical device.

FIG. 20 is a plan view showing a pulling shaft according to a ninth modification of the medical device.

### DETAILED DESCRIPTION

Set forth below with reference to the accompanying drawings is a detailed description of embodiments of a medical device that applies energy to biological tissue. Note that since embodiments described below are preferred specific examples of the present disclosure, although various technically preferable limitations are given, the scope of the present disclosure is not limited to the embodiments unless otherwise specified in the following descriptions. Dimensional ratios in the drawings may be exaggerated and different from actual ratios for convenience of description. In the present specification, a side on which a medical device 10 is inserted into a biological lumen will be referred to as a “distal side”, and an operation side will be referred to as a “proximal side”.

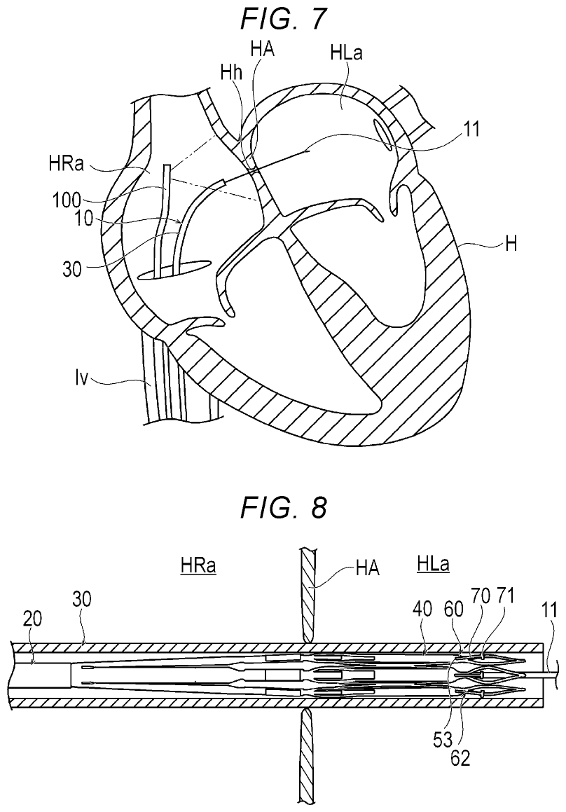

As shown in FIG. 7, a medical device according to the present embodiment is configured to enlarge a through-hole Hh formed in an atrial septum HA of a patient's heart H, and then perform a maintenance treatment to maintain the enlarged through-hole Hh at an increased size.

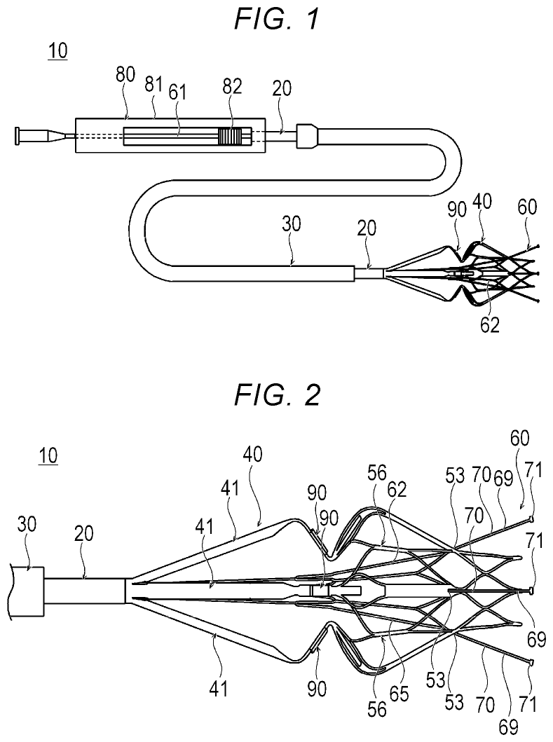

As shown in FIGS. 1 and 2, the medical device 10 according to the present embodiment can include an elongated outer tube 20, a storage sheath 30 that stores the outer tube 20, an expansion body 40 provided at a distal portion of the outer tube 20, and a pulling shaft 60 that pulls the expansion body 40. The medical device 10 can further include an operation unit 80 provided at a proximal portion of the outer tube 20, and an energy transfer element 90 that is disposed in the expansion body 40 and performs the maintenance treatment described above.

The distal portion of the outer tube 20 is fixed to a proximal portion of the expansion body 40. The proximal portion of the outer tube 20 is fixed to the operation unit 80.

The storage sheath 30 is movable forward and backward with respect to the outer tube 20 in an axial direction (a direction along an axis). The storage sheath 30 can store the expansion body 40 in the storage sheath 30 while having moved to a distal side of the outer tube 20. The storage sheath 30 can expose the expansion body 40 by moving to a proximal side from a state in which the expansion body 40 is stored.

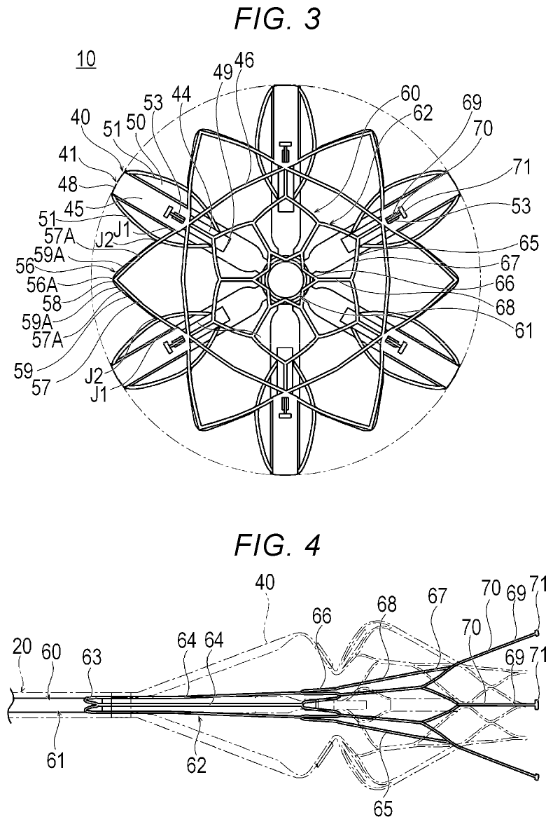

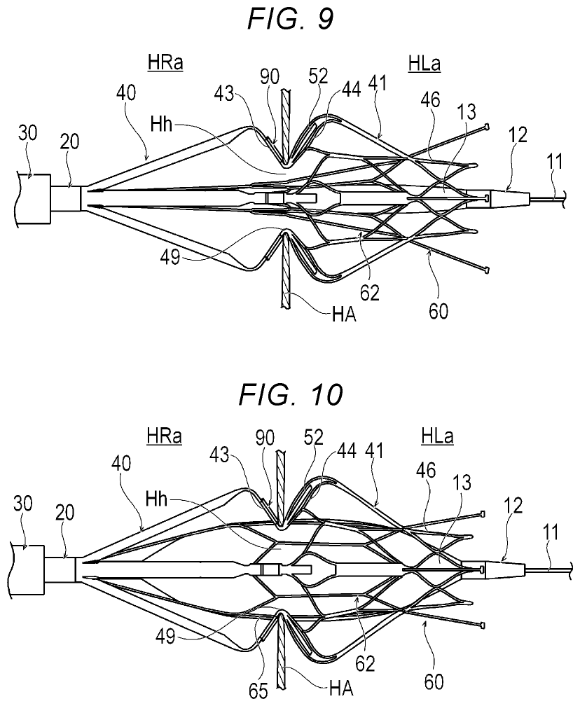

As shown in FIGS. 2 to 4, the pulling shaft 60 can include a pulling tube 61 movable forward and backward in the axial direction inside the outer tube 20, and a spreading portion 62 fixed to a distal portion of the pulling tube 61. A proximal portion of the pulling tube 61 is drawn outward from the operation unit 80 to the proximal side. A lumen is formed in the pulling tube 61 along the axial direction, and a guide wire 11 and a balloon catheter 12 (see FIGS. 9 to 11) can be inserted through the lumen.

The spreading portion 62 is movable inside the expansion body 40 along an axis of the expansion body 40. The spreading portion 62 can include a proximal connection portion 63 fixed to the distal portion of the pulling tube 61, a plurality of proximal wires 64 extending from the proximal connection portion 63 toward a distal direction, a link portion 65 extending from the proximal wires 64 toward the distal direction to connect the proximal wires 64 to each other, and a plurality of sub-wires 69 extending from the link portion 65 toward the distal direction. At least a part of the spreading portion 62 is located on the distal side of the outer tube 20.

The plurality of proximal wires 64 are arranged at equal intervals in a circumferential direction around the axis of the expansion body 40. The number of the proximal wires 64 is not particularly limited, and can be, for example, six.

The link portion 65 connects circumferentially adjacent proximal wires 64 to each other, and connects circumferentially adjacent sub-wires 69 to each other. The link portion 65 can be formed in a honeycomb structure in which a plurality of hexagonal frames are arranged while being connected in the circumferential direction around the axis of the expansion body 40. The number of hexagonal frames can be, for example, six corresponding to the number of proximal wires 64 and sub-wires 69. The number of hexagonal frames is not particularly limited.

The link portion 65 can include a proximal link portion 66 connected to distal portions of the proximal wires 64, a distal link portion 67 connected to proximal portions of the sub-wires 69, and a plurality of intermediate link portions 68 provided between the distal link portion 67 and the proximal link portion 66.

The proximal link portion 66 can be formed in an annular shape around the axis of the expansion body 40 while being folded back in a zigzag manner toward the distal side and the proximal side so as to be alternately connected to proximal portions of the intermediate link portions 68 and the distal portions of the proximal wires 64.

The distal link portion 67 is formed in an annular shape around the axis of the expansion body 40 while being folded back in a zigzag manner toward the distal side and the proximal side so as to be alternately connected to distal portions of the intermediate link portions 68 and the proximal portions of the sub-wires 69.

The intermediate link portions 68 can be arranged at equal intervals in the circumferential direction around the axis of the expansion body 40. Each of the intermediate link portions 68 extends along the axis of the expansion body 40. The proximal portion of the intermediate link portion 68 is connected to a portion of the proximal link portion 66 that protrudes toward the distal direction, and the distal portion of the intermediate link portion 68 is connected to a portion of the distal link portion 67 that protrudes toward a proximal direction. Therefore, connection portions between the intermediate link portions 68 and the proximal link portion 66 and connection portions between the intermediate link portions 68 and the distal link portion 67 are not caught by other members when sliding with respect to other members along the axis.

The link portion 65 formed in the honeycomb structure has a tubular shape, and can expand and contract in a radial direction by changing an angle of a corner of a hexagon. The link portion 65 may not be formed in the honeycomb structure in which hexagons are arranged, and may be formed in a lattice structure in which rhombuses are arranged, for example.

The plurality of sub-wires 69 are arranged at equal intervals in the circumferential direction around the axis of the expansion body 40. The number of the sub-wires 69 is not particularly limited, and can be, for example, six. Each of the sub-wires 69 includes a linear sliding shaft 70 and an engagement portion 71 disposed at a distal portion of the sliding shaft 70. The sliding shaft 70 can be slidable with respect to the expansion body 40. The engagement portion 71 can be engaged with the expansion body 40 in order to pull the expansion body 40 toward the proximal direction. For example, the engagement portion 71 is formed in a T-shape at the distal portion of the sliding shaft 70, and protrudes in two directions perpendicular to the axis of the expansion body 40 when viewed from a radially outer side. A shape of the engagement portion 71 is not particularly limited as long as the engagement portion 71 can be engaged with the expansion body 40.

The spreading portion 62 is formed such that an inner diameter and an outer diameter are increased from a proximal portion toward the distal direction in the whole or at least a part of the spreading portion 62. The proximal portion of the spreading portion 62 can be accommodated in the outer tube 20. A portion of the spreading portion 62 on the distal side of the portion accommodated in the outer tube 20 spreads outward in the radial direction than an inner diameter of the outer tube 20. Since the spreading portion 62 is formed in a net shape, the spreading portion 62 can expand and contract in the radial direction. The spreading portion 62 can be formed by applying laser processing to a circular tube that is a material, which can be used for the spreading portion 62. A method for forming the spreading portion 62 is not limited to applying laser processing to a circular tube.

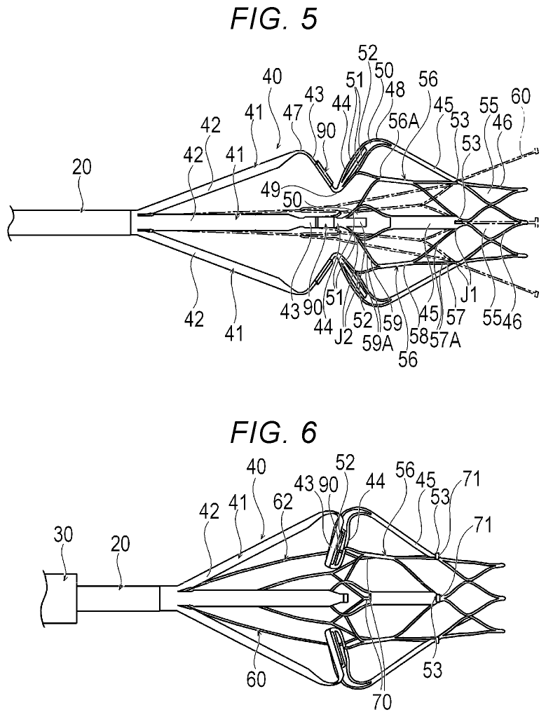

As shown in FIGS. 2, 3 and 5, the expansion body 40 includes a plurality of main struts 41 arranged in the circumferential direction around the axis of the expansion body 40, and a plurality of sub-struts 56 arranged between circumferentially adjacent main struts 41. The main struts 41 and the sub-struts 56 are alternately arranged in the circumferential direction. The number of the main struts 41 and the number of the sub-struts 56 are not particularly limited, and the number of the main struts 41 and the number of the sub-struts 56 are each, for example, six. A strut means a columnar member capable of supporting a load.

Each of the main struts 41 can expand and contract in the radial direction of the expansion body 40. The expansion body 40 deploys in the radial direction in a natural state in which no external force acts. A proximal portion of the main strut 41 is fixed to the distal portion of the outer tube 20. The main strut 41 can include a proximal side main strut 42, a proximal side sandwiching strut 43, a distal side sandwiching strut 44, a distal side main strut 45, and a distal side connection strut 46. The main strut 41 has the following shape in a deployed form.

The proximal side main strut 42 is inclined so as to increase in the radial direction from the proximal portion of the expansion body 40 toward the distal direction. The distal side main strut 45 can be inclined so as to increase in the radial direction toward the proximal direction from the distal side connection strut 46 located at a distal portion of the expansion body 40. Each of the proximal side main strut 42 and the distal side main strut 45 extends linearly.

The proximal side sandwiching strut 43 can be inclined so as to decrease in the radial direction from a distal portion of the proximal side main strut 42 toward the distal direction. The proximal side sandwiching strut 43 and the proximal side main strut 42 are connected to each other by a proximal side outward protruding portion 47 protruding outward in the radial direction. The distal side sandwiching strut 44 is inclined so as to decrease in the radial direction from a proximal portion of the distal side main strut 45 toward the proximal direction. The distal side sandwiching strut 44 and the distal side main strut 45 are connected to each other by a distal side outward protruding portion 48 protruding outward in the radial direction. The proximal side sandwiching strut 43 and the distal side sandwiching strut 44 are connected to each other by an inward protruding portion 49 protruding inward in the radial direction. An axial interval between the proximal side sandwiching strut 43 and the distal side sandwiching strut 44 can be preferably slightly larger on an outer side than on an inner side in the radial direction in the deployed form. Accordingly, it can be relatively easy to dispose biological tissue between the proximal side sandwiching strut 43 and the distal side sandwiching strut 44 from the radially outer side.

In the main strut 41, one intermediate through-hole 50 is formed in the vicinity of the proximal portion of the distal side main strut 45 and the distal side sandwiching strut 44. The intermediate through-hole 50 penetrates in the radial direction of the expansion body 40. The main strut 41 includes two outer edge portions 51 sandwiching the intermediate through-hole 50 and a backing portion 52 provided between the two outer edge portions 51. The backing portion 52 can face the energy transfer element 90 disposed on the proximal side sandwiching strut 43 when the backing portion 52 contracts in the direction along the axis of the expansion body 40. Each of the outer edge portions 51 can have an arc shape in the deployed form. Therefore, a relatively wide region for disposing the backing portion 52 and forming the intermediate through-hole 50 can be ensured between the two outer edge portions 51.

The backing portion 52 protrudes from a portion of the distal side sandwiching strut 44 on an inward protruding portion 49 side toward a proximal portion of the distal side sandwiching strut 44, between the two outer edge portions 51. The backing portion 52 is disposed between the two outer edge portions 51 at an interval from the two outer edge portions 51. The backing portion 52 can have a cantilever shape in which a proximal portion is fixed, and is thus relatively easily bent. Therefore, the backing portion 52 can be more easily bent than the outer edge portion 51 by a force toward the distal side received from the energy transfer element 90 disposed on the proximal side sandwiching strut 43.

A force reception portion 53 that slidably holds the sliding shaft 70 of the pulling shaft 60 is formed in a distal portion of the distal side main strut 45. The force reception portion 53 can be a rectangular hole having a long side in the axial direction of the expansion body 40. Therefore, a direction of the long side of the force reception portion 53 is substantially perpendicular to the direction of the T-shaped engagement portion 71 of the pulling shaft 60. Therefore, the force reception portion 53 is engaged with the engagement portion 71 without enabling the engagement portion 71 to pass through the force reception portion 53 while slidably holding the sliding shaft 70. The force reception portion 53 can receive a pulling force from the engagement portion 71 by being engaged with the engagement portion 71. The T-shaped engagement portion 71 of the sub-wire 69 can be inserted into the force reception portion 53 by intentionally twisting the sub-wire 69 by, for example, 90 degrees. The plurality of sub-wires 69 arranged in the circumferential direction are connected by the link portion 65, and are thus less likely to be twisted. Therefore, when the sub-wire 69 is intentionally twisted by, for example, 90 degrees to insert the T-shaped engagement portion 71 into the force reception portion 53 and then the sub-wire 69 returns from twisting, the engagement portion 71 cannot pass through the force reception portion 53. A position at the main strut 41 where the force reception portion 53 is formed is located radially outward than the radially innermost surface of the inward protruding portion 49.

The distal side connection strut 46 is located at a distal portion of the main strut 41. The plurality of distal side connection struts 46 are arranged in an annular shape and connected in the circumferential direction. Each of the distal side connection struts 46 has a substantially rhombic distal through-hole 55 penetrating in the radial direction of the expansion body 40, and can be formed in a substantially rhombic frame shape. That is, each of the distal side connection struts 46 is formed in a lattice structure that can be changed into a quadrangle having the same length on all four sides but different angles. The plurality of distal side connection struts 46 are arranged in the annular shape and connected in the circumferential direction by joining opposite points of each rhombus. Therefore, the plurality of distal side connection struts 46 arranged in the annular shape are connected to each other so as to be expandable and contractible in the radial direction by using the lattice structure. Therefore, the position of the force reception portion 53 that slidably holds the pulling shaft 60 is movable in the radial direction.

Each of the sub-struts 56 can be disposed between two circumferentially adjacent main struts 41, and is connected to the two main struts 41. Each of the sub-struts 56 includes a proximal side support strut 59 (support strut) connected to two circumferentially adjacent outer edge portions 51, a distal side support strut 57 (support strut) connected to distal portions of two circumferentially adjacent distal side main struts 45, and a merging strut 58 provided between the proximal side support strut 59 and the distal side support strut 57.

Each of the distal side support struts 57 includes two distal side inclined struts 57A and a merging portion connecting the two distal side inclined struts 57A. Each of the two distal side inclined struts 57A extends toward the proximal direction from a joint portion J1 with the distal portion of the main strut 41 so as to be inclined with respect to the axis of the expansion body 40 when viewed from the radially outer side, and is connected to a distal portion of the merging strut 58. The two distal side inclined struts 57A connected to the same merging strut 58 have a plane-symmetrical shape with respect to a plane passing through the merging portion of the two distal side inclined struts 57A and the axis of the expansion body 40. In the deployed form, each of the distal side support struts 57 is formed to be longer than a linear distance between joint portions J1 with two main struts 41 connected with the distal side support struts 57 when viewed from the radially outer side. Therefore, when the expansion body 40 is in an expansion form in which the expansion body 40 expands in the radial direction from the deployed form, each of the distal side support struts 57 can be deformed so as to be close to a linear shape such that the two joint portions J1 are separated from each other.

Each of the proximal side support struts 59 includes two proximal side inclined struts 59A. Each of the two proximal side inclined struts 59A extends toward the distal direction from a joint portion J2 with the outer edge portion 51 of the main strut 41 so as to be inclined with respect to the axis of the expansion body 40 when viewed from the radially outer side, and is connected to a proximal portion of the merging strut 58. The two proximal side inclined struts 59A connected to the same merging strut 58 have a plane-symmetrical shape with respect to a plane passing through the merging portion of the two proximal side inclined struts 59A and the axis of the expansion body 40. In the deployed form, each of the proximal side support struts 59 is formed to be longer than a linear distance between joint portions J2 with two main struts 41 connected with the proximal side support struts 59 when viewed from the radially outer side. Therefore, when the expansion body 40 is in the expansion form in which the expansion body 40 expands in the radial direction from the deployed form, each of the proximal side support struts 59 can be deformed so as to be close to a linear shape such that the two joint portions J2 are separated from each other.

The merging struts 58 are arranged at equal intervals in the circumferential direction around the axis of the expansion body 40. Each of the merging strut 58 extends between the distal side support strut 57 and the proximal side support strut 59 so as to be substantially parallel to the axis of the expansion body 40 when viewed from the radially outer side. In the sub-strut 56, a sub-strut outward protruding portion 56A protruding outward in the radial direction is formed in the proximal side support strut 59 or the merging strut 58.

In a cross section, of a portion where the sub-strut 56 is present, perpendicular to the axis at any position in the axial direction, a radially outermost position of the main strut 41 of the expansion body 40 in the natural state is located radially outward than a radially outermost position of the sub-strut 56. Further, in a cross section, of the expansion body 40 in the natural state at a position where the distal side outward protruding portion 48 is provided, perpendicular to the axis, the distal side outward protruding portion 48 of the main strut 41 is located radially outward than a radially outermost position of the sub-strut 56.

As shown in FIG. 6, when the pulling shaft 60 moves to the proximal side, the sliding shaft 70 slides along the force reception portion 53, and the engagement portion 71 is engaged with the force reception portion 53. The engagement portion 71 engaged with the force reception portion 53 can apply a pulling force toward the proximal direction to the force reception portion 53. Accordingly, the expansion body 40 is compressed in the axial direction and can be in the expansion form in which the expansion body 40 expands in the radial direction from the deployed form. When the expansion body 40 is in the expansion form, the proximal side sandwiching strut 43 and the distal side sandwiching strut 44 approach each other.

The main struts 41 and the sub-struts 56 constituting the expansion body 40 are integrally formed by applying laser processing to a cylinder, for example. The main strut 41 and the sub-strut 56 may each have a thickness, for example, of 50 μm to 500 μm and a width, for example, of 0.1 mm to 2.0 mm. However, the main strut 41 and the sub-strut 56 may have dimensions out of this range. Shapes of the main strut 41 and the sub-strut 56 are not limited, and the main strut 41 and the sub-strut 56 may each have, for example, a circular cross-sectional shape or other cross-sectional shapes.

As shown in FIGS. 2 and 9, the energy transfer element 90 is disposed on the proximal side sandwiching strut 43 so as to face the backing portion 52 of the distal side sandwiching strut 44. Therefore, when the proximal side sandwiching strut 43 and the distal side sandwiching strut 44 sandwiches the atrial septum HA, energy from the energy transfer element 90 is transferred to the atrial septum HA from a right atrium side. The energy transfer element 90 may be disposed in the distal side sandwiching strut 44, and the backing portion 52 may be disposed on the proximal side sandwiching strut 43. In this case, the energy from the energy transfer element 90 is transferred to the atrial septum HA from a left atrium side.

For example, the energy transfer element 90 can include a bipolar electrode that receives electric energy from an energy supply device that is an external device. In this case, electricity is conducted between the energy transfer elements 90 disposed in the respective main struts 41. The energy transfer element 90 and the energy supply device are connected by a conductive wire coated with an insulating coating material. The conductive wire can extend outward via the outer tube 20 and the operation unit 80 so as to be connected to the energy supply device.

Alternatively, the energy transfer element 90 may be configured as a monopolar electrode. In this case, the electricity is conducted between the energy transfer element 90 and a counter electrode plate prepared outside a body. The energy transfer element 90 may be a heating element (electrode chip) that generates heat by receiving high-frequency electric energy from the energy supply device. In this case, the electricity is conducted between the energy transfer elements 90 disposed in the respective main struts 41. The energy transfer element 90 may include an element capable of applying energy to the through-hole Hh, such as a heater including an electric wire, which provides heating and cooling operation or generates frictional heat using microwave energy, ultrasonic energy, coherent light such as laser, a heated fluid, a cooled fluid, or a chemical medium. A specific form of the energy transfer element 90 is not particularly limited.

As shown in FIG. 1, the operation unit 80 includes a housing 81 to be held by an operator and a moving unit 82 operable by the operator. The moving unit 82 is fixed to the pulling shaft 60 inside the operation unit 80. The moving unit 82 is movable forward and backward with respect to the housing 81 in the axial direction of the pulling shaft 60. Therefore, the operator can move the pulling shaft 60 in the axial direction by moving the moving unit 82.

The expansion body 40 can be formed of a metal material. Examples of the metal material include a titanium-based (Ti—Ni, Ti—Pd, Ti—Nb—Sn, or the like) alloy, a copper-based alloy, stainless steel, p-titanium steel, and a Co—Cr alloy. It is more preferable to use an alloy or the like having a spring property such as a nickel-titanium alloy. However, a material of the expansion body 40 is not limited to a metal material, and the expansion body 40 may be formed of other materials.

The storage sheath 30 and the outer tube 20 are preferably formed of a material having a certain degree of flexibility. Examples of the materials for the storage sheath 30 and the outer tube 20 can include polyolefins such as polyethylene, polypropylene, polybutene, ethylene-propylene copolymer, ethylene-vinyl acetate copolymer, ionomer, or a mixture of two or more of these, fluororesin such as soft polyvinyl chloride resin, polyamide, polyamide elastomer, polyester, polyester elastomer, polyurethane, or polytetrafluoroethylene, polyimide, PEEK, silicone rubber, and latex rubber.

For example, the pulling tube 61 can be formed of a coil-shaped member formed by winding an elongated wire or plate material made of a super-elastic alloy such as a nickel-titanium alloy or a copper-zinc alloy, or a metal material such as stainless steel, a pipe with slits made of these metal materials, or a tube body made of a resin material having relatively high rigidity. The pulling tube 61 may include an outer coating layer in which an outer peripheral surface of the pulling tube 61 is coated with a resin material such as polyvinyl chloride, polyethylene, polypropylene, ethylene-propylene copolymer, or fluororesin. Accordingly, the pulling tube 61 rather easily moves forward and backward in the axial direction inside the outer tube 20. The pulling tube 61 may include an inner coating layer in which an inner peripheral surface of the pulling tube 61 is coated with the above-described resin material (in particular, the fluororesin). Accordingly, the guide wire 11 and the balloon catheter 12 can be rather easily inserted into the pulling tube 61.

The spreading portion 62 can be formed of, for example, a super-elastic alloy such as a nickel-titanium alloy or a copper-zinc alloy, a metal material such as stainless steel, or a resin material having relatively high rigidity.

Next, a treatment method using the medical device 10 according to the present embodiment will be described. This treatment method is performed on a patient suffering from a heart failure (left heart failure). More specifically, as shown in FIG. 7, the treatment method is performed on the patient suffering from a chronic heart failure, who has high blood pressure in a left atrium HLa due to myocardial hypertrophy appearing in a left ventricle of the heart H and increased stiffness (hardness).

When the through-hole Hh is formed, the operator delivers an introducer, in which a guiding sheath and a dilator are combined with each other, to the vicinity of the atrial septum HA. For example, the introducer can be delivered to a right atrium HRa via an inferior vena cava Iv. The introducer can be delivered using the guide wire 11. The operator can insert the guide wire 11 into the dilator and deliver the introducer along the guide wire 11. The introducer and the guide wire 11 can be inserted into a living body using a method such as using an introducer for blood vessel introduction.

Next, the operator causes a puncture device and the dilator to penetrate from a right atrium HRa side toward a left atrium HLa side, thereby forming the through-hole Hh. For example, a device such as a wire having a sharp distal end can be used as the puncture device. The puncture device is inserted into the dilator and delivered to the atrial septum HA. After the guide wire 11 is removed from the dilator, the puncture device can be delivered to the atrial septum HA in place of the guide wire 11.

Next, the operator delivers the medical device 10 to the vicinity of the atrial septum HA along the guide wire 11 inserted into the left atrium HLa from the right atrium HRa via the through-hole Hh in advance. A part of a distal portion of the medical device 10 passes through the through-hole Hh formed in the atrial septum HA and reaches the left atrium HLa. As shown in FIG. 8, when the medical device 10 is inserted, the expansion body 40 is in a contraction form of being stored in the storage sheath 30. In the contraction form, the expansion body 40 and the spreading portion 62 that protrude outward in the radial direction in the natural state (deployed form) are deformed so as to contract in the radial direction, and are stored in the storage sheath 30. When the expansion body 40 is stored in the storage sheath 30, the engagement portion 71 of the pulling shaft 60 is disposed away from the force reception portion 53 of the expansion body 40 toward the distal side of the force reception portion 53. Accordingly, when the expansion body 40 contracts in the radial direction and extends in the axial direction, the force reception portion 53 of the expansion body 40 slides along the sliding shaft 70 of the pulling shaft 60 and does not come into contact with the engagement portion 71. Therefore, the pulling shaft 60 does not interfere with deformation of the expansion body 40.

Next, as shown in FIG. 9, the storage sheath 30 is moved to the proximal side to expose a distal side portion of the expansion body 40 into the left atrium HLa. Accordingly, the distal side portion of the expansion body 40 deploys in the radial direction inside the left atrium HLa by its own restoring force. Since the main strut 41 on a distal side of the inward protruding portion 49 of the expansion body 40 is supported by the sub-strut 56, the main strut 41 is less likely to be twisted in the circumferential direction. Therefore, the distal side portion of the expansion body 40 that is first released from the storage sheath 30 can deploy into an appropriate shape. Next, the entire expansion body 40 is exposed by moving the storage sheath 30 to the proximal side. Accordingly, a proximal side portion of the expansion body 40 deploys in the radial direction inside the right atrium HRa by its own restoring force. Since the distal side portion of the expansion body 40 that has deployed first has the appropriate shape by providing the sub-strut 56, the proximal side portion of the expansion body 40 that deploys later is also supported by the distal side portion and can have an appropriate shape. When the entire expansion body 40 deploys (expands), the inward protruding portion 49 is disposed inside the through-hole Hh. Accordingly, the entire expansion body 40 is deployed by its own restoring force, and is restored to the original deployed form or a form close to the deployed form. At this time, the atrial septum HA is disposed between the proximal side sandwiching strut 43 and the distal side sandwiching strut 44. The atrial septum HA is disposed between the energy transfer element 90 and the backing portion 52 in a direction in which biological tissue is sandwiched.

Next, the operator inserts the balloon catheter 12 into the lumen from a proximal side of the pulling tube 61. The balloon catheter 12 includes a balloon 13 (auxiliary expansion body) which is inflated by being supplied with a fluid, at a distal portion of an elongated tubular body. The operator causes the balloon 13 to reach a range in which the expansion body 40 is provided in the axial direction. The balloon 13 is disposed inside the inward protruding portion 49 of the expansion body 40, that is, inside the through-hole Hh. The distal side connection strut 46 located at the distal portion of the expansion body 40 expands in the radial direction by changing from the contraction form to the deployed form. Therefore, the balloon 13 can be disposed inside the distal portion of the expansion body 40. The spreading portion 62 of the pulling shaft 60 is disposed radially outward than the inner diameter of the outer tube 20. The spreading portion 62 is expandable outward in the radial direction. Therefore, the spreading portion 62 does not come into contact with the balloon 13 inserted into inside of the expansion body 40, or can be deformed so as to move outward in the radial direction even if the spreading portion 62 comes into contact with the balloon 13. Therefore, the pulling shaft 60 does not interfere with arrangement of the balloon 13 inside the expansion body 40.

Next, as shown in FIG. 10, the operator supplies the fluid for inflation to the balloon catheter 12 from the proximal side to inflate the balloon 13. At this time, the distal side connection strut 46 located at the distal portion of the expansion body 40 expands in the radial direction by changing from the contraction form to the deployed form. The spreading portion 62 of the pulling shaft 60 does not come into contact with the balloon 13 inserted into the inside of the expansion body 40, or can be deformed so as to move outward in the radial direction even if the spreading portion 62 comes into contact with the balloon 13. Accordingly, the expansion body 40 and the pulling shaft 60 do not interfere with inflation of the balloon 13 inside the expansion body 40. The inflated balloon 13 enlarges the through-hole Hh together with the inward protruding portion 49 inside the through-hole Hh.

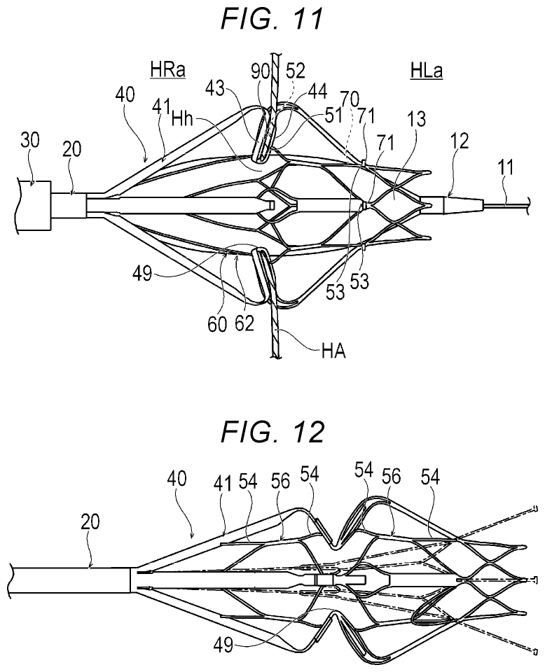

The pulling shaft 60 can move in the axial direction without being interfered by the inflated balloon 13. The pulling shaft 60 is disposed such that the inward protruding portion 49 is directed toward a hexagonal gap of the link portion 65 so that the pulling shaft 60 can move in a state where the balloon 13 is inflated. Accordingly, when the pulling shaft 60 moves, the inward protruding portion 49 of the expansion body 40 can be prevented from coming into contact with the pulling shaft 60 and interfering with movement of the pulling shaft 60. Therefore, the operator can expand the expansion body 40 by moving the pulling shaft 60 toward the proximal direction in a state where the balloon 13 is inflated. The operator operates the operation unit 80 to move the pulling shaft 60 to the proximal side. Accordingly, as shown in FIG. 11, the sliding shaft 70 slides along the force reception portion 53, and the engagement portion 71 is engaged with the force reception portion 53. The engagement portion 71 engaged with the force reception portion 53 applies a pulling force toward the proximal direction to the force reception portion 53. Accordingly, the expansion body 40 is compressed in the axial direction and is brought into the expansion form in which the expansion body 40 expands in the radial direction more than the deployed form. In the expansion form of the expansion body 40, the proximal side sandwiching strut 43 and the distal side sandwiching strut 44 approach each other, and the atrial septum HA is sandwiched between the proximal side sandwiching strut 43 and the distal side sandwiching strut 44. At this time, the energy transfer element 90 and the backing portion 52 face each other. The pulling shaft 60 is further pulled in a state in which the atrial septum HA is sandwiched between the proximal side sandwiching strut 43 and the distal side sandwiching strut 44. Accordingly, the proximal side sandwiching strut 43 and the distal side sandwiching strut 44 further expands, and the through-hole Hh can be further enlarged in the radial direction. That is, the operator can enlarge the through-hole Hh in the radial direction in conjunction with expansion of the expansion body 40 and inflation of the balloon 13. Therefore, even when the through-hole Hh, which is the tissue to be enlarged, is relatively hard, the expansion body 40 and the balloon 13 can enlarge the through-hole Hh to a desired size. After the proximal side sandwiching strut 43 and the distal side sandwiching strut 44 sandwich the atrial septum HA, the pulling shaft 60 may not be further pulled.

The main strut 41 receiving the pulling force from the pulling shaft 60 sandwiches the atrial septum HA. At this time, the main strut 41 is supported by circumferentially adjacent proximal side support struts 59 and distal side support struts 57.

Each of the distal side support struts 57 is formed to be longer than the linear distance between the two joint portions J1 in the deployed form before expansion when viewed from the radially outer side. Therefore, when the expansion body 40 is in the expansion form, each of the distal side support struts 57 can be rather easily deformed such that the two joint portions J1 are separated from each other. Therefore, the distal side support strut 57 can support the main strut 41 without applying an excessive pulling force to the main strut 41.

Each of the proximal side support struts 59 is formed to be longer than the linear distance between the two joint portions J2 in the deployed form before expansion when viewed from the radially outer side. Therefore, when the expansion body 40 is in the expansion form, each of the proximal side support struts 59 can be rather easily deformed such that the two joint portions J2 are separated from each other. Therefore, the proximal side support strut 59 can support the main strut 41 without applying an excessive pulling force to the main strut 41.

Therefore, the main strut 41 can be prevented from being twisted in the circumferential direction. Since the sub-strut 56 is located more radially inward than the main strut 41, the linear main strut 41 can be prevented from being pulled and bent by the sub-strut 56 during expansion. Therefore, in the main strut 41, a force for pressing the energy transfer element 90 against the tissue is less likely to be dispersed, and the energy transfer element 90 can be effectively pressed against the tissue.

Here, the balloon 13 is inflated and then the expansion body 40 performs sandwiching. However, the balloon 13 may be inflated after the expansion body 40 performs sandwiching.

When the atrial septum HA is sandwiched between the proximal side sandwiching strut 43 and the distal side sandwiching strut 44, the energy transfer element 90 presses the atrial septum HA toward the distal side. At this time, the distal side sandwiching strut 44 bends the backing portion 52 toward the distal side between the two outer edge portions 51, and receives the atrial septum HA pressed by the energy transfer element 90 between the two outer edge portions 51. The two outer edge portions 51 effectively guide the energy transfer element 90 to the backing portion 52 located between the outer edge portions 51. The backing portion 52 receives a force from the energy transfer element 90 via the atrial septum HA, and is bent so as to be substantially parallel to the energy transfer element 90. Then, the backing portion 52 applies a repulsive force in a direction opposite to a pressing direction of the energy transfer element 90 to the atrial septum HA pressed by the energy transfer element 90, while being flexibly bent. Accordingly, the energy transfer element 90 comes into close contact with the atrial septum HA.

The operator can confirm hemodynamics by enlarging the through-hole Hh and then deflating the balloon 13. The operator delivers a hemodynamics confirmation device 100 to the right atrium HRa via the inferior vena cava Iv. For example, an echo catheter can be used as the hemodynamics confirmation device 100. The operator can display an echo image acquired by the hemodynamics confirmation device 100 on a display device such as a display, and confirm a blood volume passing through the through-hole Hh based on the display result.

Next, the operator performs a maintenance treatment to maintain the size of the through-hole Hh. In the maintenance treatment, energy is applied to an edge portion of the through-hole Hh through the energy transfer element 90, thereby cauterizing (heating and cauterizing) the edge portion of the through-hole Hh by the energy. When the biological tissue in the vicinity of the edge portion of the through-hole Hh is cauterized through the energy transfer element 90, a degenerated portion having the degenerated biological tissue is formed in the vicinity of the edge portion. Since the biological tissue in the degenerated portion loses elasticity, the through-hole Hh can maintain a shape when being enlarged by the expansion body 40 and the balloon 13.

After the maintenance treatment, the operator discharges the fluid for inflation from the balloon 13 to deflate the balloon 13, and then confirms the hemodynamics again. When the blood volume passing through the through-hole Hh is a desired volume, the operator removes the balloon catheter 12 from the medical device 10. Next, the operator reduces a diameter of the expansion body 40, stores the expansion body 40 in the storage sheath 30, and then removes the expansion body 40 from the through-hole Hh. Further, the operator removes the entire medical device 10 outward of the living body, and ends the treatment.

As described above, the medical device 10 according to the present embodiment includes: the elongated outer tube 20; the expansion body 40 connected to a distal portion of the outer tube 20 and configured to expand in a radial direction by contracting along an axis of the outer tube 20; the pulling shaft 60 configured to slide with respect to the outer tube 20, the pulling shaft 60 being disposed inside the outer tube 20, protruding from the distal portion of the outer tube 20, and being connected to a distal portion of the expansion body 40; and the plurality of energy transfer elements 90 disposed in the expansion body 40 and configured to output energy. The expansion body 40 includes the plurality of main struts 41 arranged at intervals in the circumferential direction and extending along the axis of the outer tube 20 by a predetermined length, and the plurality of sub-struts 56 connected to the plurality of main struts 41. At least one of the plurality of energy transfer elements 90 is disposed on each of the plurality of main struts 41. Each of the plurality of main struts 41 includes the force reception portion 53 configured to receive a pulling force from the pulling shaft 60. A portion of each of the plurality of main struts 41 between the force reception portion 53 and the energy transfer element 90 is substantially parallel to the axis when viewed from a radially outer side. Each of the plurality of sub-struts 56 includes the distal side support strut 57 and the proximal side support strut 59, and the distal side support strut 57 and the proximal side support strut 59 each have two joint portions joined to two circumferentially adjacent main struts 41 among the plurality of main struts 41. The distal side support strut 57 and the proximal side support struts 59 are each formed longer than the linear distance between the two joint portions.

In the medical device 10 configured as described above, the distal side support strut 57 and the proximal side support strut 59 helps prevent the main strut 41 receiving the pulling force from being twisted in the circumferential direction when the energy transfer element 90 is pressed against tissue. Therefore, in the medical device 10, a force for pressing the energy transfer element 90 against the tissue is less likely to be dispersed, and the energy transfer element 90 can be effectively pressed against the biological tissue.

Each of the plurality of support struts (distal side support strut 57 or proximal side support strut 59) includes the two inclined struts (distal side inclined strut 57A or proximal side inclined strut 59A) respectively extending from two circumferentially adjacent main struts 41 so as to be inclined with respect to the axis when viewed from the radially outer side, and the merging portion connecting the two inclined struts to each other, and the two inclined struts connected to the merging portion are plane-symmetrical with respect to a plane passing through the merging portion and the axis of the expansion body 40. Accordingly, when the expansion body 40 is deformed, the two inclined struts that are plane-symmetrical are deformed into a symmetrical shape. Therefore, forces acting on the two circumferentially adjacent main struts 41 from the inclined struts are equal to each other. Therefore, the main strut 41 can be prevented from being twisted in the circumferential direction. Even if the inclined strut has a curved shape, the inclined strut can be regarded as an inclined strut because a tangent line of any part is inclined with respect to the axis.

The distal side support struts 57 and the proximal side support struts 59 are disposed at a plurality of positions in an axial direction of the expansion body 40. Accordingly, in the medical device 10, when the energy transfer element 90 is pressed against the tissue, a twist of the main strut 41 in the circumferential direction can be effectively prevented by the plurality of distal side support struts 57 and proximal side support struts 59 in the axial direction.

The distal side support strut 57 and the proximal side support strut 59 disposed at the plurality of positions in the axial direction of the expansion body 40 are connected to each other. In the present embodiment, the distal side support strut 57 and the proximal side support strut 59 are connected to each other by the merging strut 58. Accordingly, in the medical device 10, when the energy transfer element 90 is pressed against the biological tissue, the twist of the main strut 41 in the circumferential direction can be effectively prevented by the distal side support strut 57 and the proximal side support strut 59 connected adjacently in the axial direction. Since the distal side support strut 57 and the proximal side support strut 59 disposed at the plurality of positions in the axial direction are connected to each other, the main strut 41 can be prevented from being bent. Therefore, in the medical device 10, the force for pressing the energy transfer element 90 against the tissue is less likely to be dispersed, and the energy transfer element 90 can be effectively pressed against the tissue.

The expansion body 40 includes the distal side sandwiching strut 44 and the proximal side sandwiching strut 43 whose separation distance is narrowed by expansion of the expansion body 40, the inward protruding portion 49 protruding inward in the radial direction is formed between the distal side sandwiching strut 44 and the proximal side sandwiching strut 43, and the support strut is disposed on at least one of a distal side and a proximal side of the inward protruding portion 49. Accordingly, the distal side sandwiching strut 44 and the proximal side sandwiching strut 43 are less likely to be twisted in the circumferential direction due to the support strut. Therefore, a force for holding the tissue by the distal side sandwiching strut 44 and the proximal side sandwiching strut 43 is less likely to be dispersed, and the tissue can be effectively held.

The disclosure is not limited to the embodiment described above, and various modifications can be made by those skilled in the art within a scope of the technical idea of the disclosure. For example, the distal side support strut 57 and the proximal side support strut 59 may be directly connected to each other without being connected by the elongated merging strut 58. In addition, the merging strut 58 may not be provided, and the distal side support strut 57 and the proximal side support strut 59 may be separately disposed.

As in a first modification shown in FIG. 12, the sub-struts 56 each including support struts 54 may be provided on both a distal side and a proximal side of the inward protruding portion 49. Alternatively, the sub-strut 56 may be provided only on the proximal side of the inward protruding portion 49.

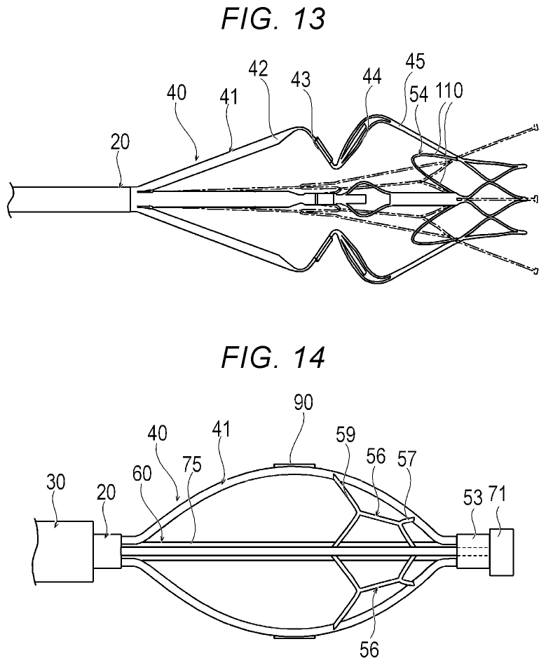

As in a second modification shown in FIG. 13, an arc-shaped support strut 54 connected to two circumferentially adjacent main struts 41 may include two inclined struts 110. A position where the arc-shaped support strut 54 is connected to the main strut 41 is not limited to the distal side main strut 45, and for example, may be the distal side sandwiching strut 44, the proximal side sandwiching strut 43, or the proximal side main strut 42.

As in a third modification shown in FIG. 14, the pulling shaft 60 may include an inner tube 75 movable inside the outer tube 20 in an axial direction, and the engagement portion 71 to which a distal portion of the inner tube 75 is fixed. The engagement portion 71 is pulled toward a proximal direction by the inner tube 75, and can compress the expansion body 40 in the axial direction. The expansion body 40 includes, at a distal portion of the expansion body 40, the force reception portion 53 having a circular tube shape to which a plurality of main struts 41 are connected. The engagement portion 71 may have a ring shape with an opening such that the guide wire 11 can be inserted through the engagement portion 71, or may have a shape without an opening. The main strut 41 of the expansion body 40 may not be provided with the distal side sandwiching strut 44 and the proximal side sandwiching strut 43, and may be expandable in a manner of being bent outward in a radial direction when pulled by the pulling shaft 60. The energy transfer element 90 is disposed in the main strut 41, but may not be disposed in the main strut 41.

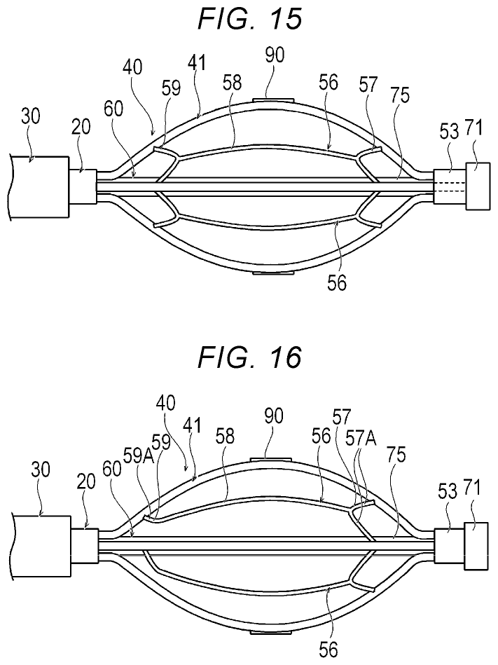

As in a fourth modification shown in FIG. 15, the distal side support strut 57 of the sub-strut 56 may be connected to a distal portion of the main strut 41, and the proximal side support strut 59 may be connected to a proximal portion of the main strut 41.

As in a fifth modification shown in FIG. 16, the distal side support strut 57 of the sub-strut 56 may include two distal side inclined struts 57A, and the proximal side support strut 59 may include only one proximal side inclined strut 59A. In addition, only one distal side inclined strut 57A may be provided, and two proximal side inclined struts 59A may be provided.

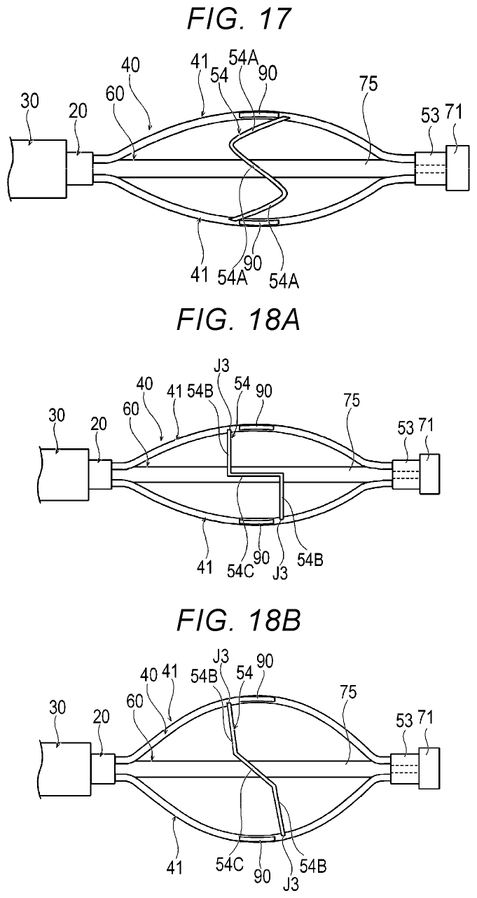

As in a sixth modification shown in FIG. 17, the support strut 54 may include three inclined struts 54A that are continuous with each other while the expansion body 40 is folded back in a zigzag manner in an axial direction.

As in a seventh modification shown in FIGS. 18A and 18B, the support strut 54 may include two first inclined struts 54B and one second inclined strut 54C between two joint portions J3 joined respectively to two circumferentially adjacent main struts 41. In a deployed form, each of the first inclined struts 54B extends from the joint portion J3 perpendicularly to an axis when viewed from a radially outer side. The second inclined strut 54C connects the two first inclined struts 54B. In the deployed form, the second inclined strut 54C is parallel to the axis when viewed from the radially outer side. Each of the support struts 54 is formed to be longer than a linear distance between the two joint portions J3 in the deployed form. When the expansion body 40 is in an expansion form, each of the support struts 54 is deformed such that the two joint portions J3 are separated from each other. At this time, the first inclined struts 54B are inclined from a state of being perpendicular to the axis when viewed from the radially outer side, and the second inclined strut 54C is inclined from a state of being parallel to the axis when viewed from the radially outer side. That is, the first inclined struts 54B and the second inclined strut 54C are inclined with respect to the axis when viewed from the radially outer side in either the deployed form or the expansion form. Even in such a form, the support strut 54 can support the main strut 41 without applying an excessive pulling force to the main strut 41.

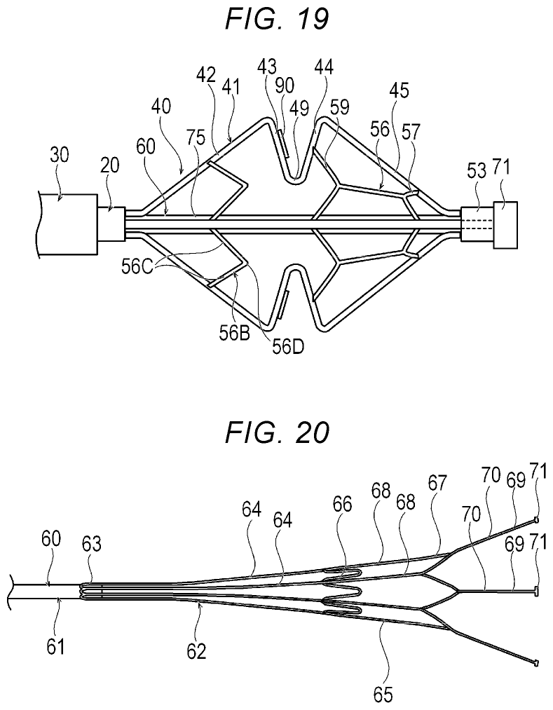

As in an eighth modification shown in FIG. 19, the expansion body 40 may include, at a distal portion of the expansion body 40, the force reception portion 53 having a circular tube shape (tubular shape) to which the plurality of main struts 41 are connected, and each of the main struts 41 may include the inward protruding portion 49. The pulling shaft 60 includes the inner tube 75 movable inside the outer tube 20 in an axial direction, and the engagement portion 71 fixed to a distal portion of the inner tube 75. The engagement portion 71 is pulled toward a proximal direction by the inner tube 75, and can compress the expansion body 40 in the axial direction. Each of the main struts 41 includes the proximal side main strut 42, the proximal side sandwiching strut 43, the distal side sandwiching strut 44, and the distal side main strut 45 from a proximal side toward a distal side.

The proximal side main strut 42 is inclined so as to increase in a radial direction from a distal portion of the outer tube 20 toward a distal direction, and the distal side main strut 45 is inclined so as to increase in the radial direction from the force reception portion 53 having the circular tube shape toward the proximal direction. The proximal side sandwiching strut 43 is inclined so as to decrease in the radial direction from a distal portion of the proximal side main strut 42 toward the distal direction, and the distal side sandwiching strut 44 is inclined so as to decrease in the radial direction from a proximal portion of the distal side main strut 45 toward the proximal direction. The proximal side sandwiching strut 43 and the distal side sandwiching strut 44 are connected to each other by the inward protruding portion 49 protruding inward in the radial direction. The energy transfer element 90 is disposed at a position where one of the proximal side sandwiching strut 43 and the distal side sandwiching strut 44 of the main strut 41 such that the energy transfer element 90 sandwiches biological tissue with the other of the proximal side sandwiching strut 43 and the distal side sandwiching strut 44 of the main strut 41.

The expansion body includes the sub-strut 56 (i.e., distal side sub-strut) on a distal side of the inward protruding portion 49, and includes a proximal side sub-strut 56B on a proximal side of the inward protruding portion 49. The distal side support strut 57 at a distal portion of the sub-strut 56 is connected to two circumferentially adjacent distal side main struts 45, and the proximal side support strut 59 at a proximal portion of the sub-strut 56 is connected to two circumferentially adjacent distal side sandwiching struts 44.

The proximal side sub-strut 56B is connected to two circumferentially adjacent proximal side main struts 42. The proximal side sub-strut 56B includes two inclined struts 56C inclined with respect to an axis when viewed from a radially outer side. The two inclined struts 56C extend toward the proximal direction while approaching the respective circumferentially adjacent proximal side main struts 42, and are connected to each other at a merging portion 56D. The two inclined struts 56C connected to the merging portion 56D are plane-symmetrical with respect to a plane passing through the merging portion 56D and the axis of the expansion body 40. In the eighth modification, since the medical device 10 includes the sub-strut 56 and the proximal side sub-strut 56B at positions separated from each other in the axial direction of the expansion body 40, the main strut 41 can be effectively prevented from being twisted in the circumferential direction when the energy transfer element 90 is pressed against the tissue.

As in a ninth modification shown in FIG. 20, the proximal wire 64 and the intermediate link portion 68 of the spreading portion 62 may be linearly arranged. The proximal link portion 66 connects connection portions of the proximal wire 64 and the intermediate link portion 68 to each other, and protrudes toward a distal direction. In this case, when the spreading portion 62 slides toward a proximal direction with respect to other members, the proximal link portion 66 is not caught by other members. In a case of the spreading portion 62 in the embodiment shown in FIG. 4, when the balloon 13 having a large expansion dimension is used, a length of the spreading portion 62 in the axial direction tends to be short, and when the balloon 13 having a small expansion dimension is used, a length of the spreading portion 62 in the axial direction tends to be long. Therefore, it is necessary to adjust a pulling amount of the pulling shaft 60 according to the deviation. In contrast, a change in a length of the spreading portion 62 in an axial direction due to expansion and contraction is small in the ninth modification. Therefore, variation in a pulling amount of the pulling shaft 60 due to an expansion dimension of the balloon 13 can be prevented.

The detailed description above describes embodiments of a medical device that applies energy to biological tissue. These disclosed embodiments represent examples of the medical device that applies energy to biological tissue disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
an elongated outer tube;
an expansion body connected to a distal portion of the outer tube and configured to expand in a radial direction;
a pulling shaft configured to slide with respect to the outer tube, the pulling shaft being disposed inside the outer tube, protruding from the distal portion of the outer tube, and being connected to a distal portion of the expansion body;
a plurality of energy transfer elements disposed in the expansion body and configured to output energy;
the expansion body including a plurality of main struts arranged at intervals in a circumferential direction and extending along an axis of the outer tube by a predetermined length, and a plurality of sub-struts connected to the plurality of main struts;
at least one of the plurality of energy transfer elements is disposed on each of the plurality of main struts;
each of the plurality of main struts includes a force reception portion configured to receive a pulling force from the pulling shaft;
a portion of each of the plurality of main struts between the force reception portion and the energy transfer element is substantially parallel to an axis of the expansion body when viewed from a radially outer side;
each of the plurality of sub-struts includes at least one support strut having two joint portions joined respectively to two circumferentially adjacent main struts among the plurality of main struts; and
each of the plurality of support struts is formed to be longer than a linear distance between the two joint portions.

2. The medical device according to claim 1, wherein
each of the plurality of support struts includes two inclined struts respectively extending from two circumferentially adjacent main struts so as to be inclined with respect to the axis of the expansion body when viewed from the radially outer side, and a merging portion connecting the two inclined struts to each other; and
the two inclined struts connected to the merging portion are plane-symmetrical with respect to a plane passing through the merging portion and the axis of the expansion body.

3. The medical device according to claim 1, wherein
the support struts are disposed at a plurality of positions in an axial direction of the expansion body.

4. The medical device according to claim 3, wherein
the plurality of support struts disposed at the plurality of positions in the axial direction of the expansion body are connected to each other.

5. The medical device according to claim 1, wherein
the expansion body includes a distal side sandwiching strut and a proximal side sandwiching strut whose separation distance is narrowed by expansion of the expansion body;
an inward protruding portion protruding inward in the radial direction is formed between the distal side sandwiching strut and the proximal side sandwiching strut; and
the support strut is disposed on at least one of a distal side and a proximal side of the inward protruding portion.

6. The medical device according to claim 5, wherein the energy transfer element is disposed at a position where one of the proximal side sandwiching strut and the distal side sandwiching strut is configured to sandwich biological tissue with an other of the proximal side sandwiching strut and the distal side sandwiching strut.

7. The medical device according to claim 6, wherein the energy transfer element is disposed on the proximal side sandwiching strut.

8. The medical device according to claim 1, wherein the expansion body is configured to expand in the radial direction by being contracted along the axis of the outer tube.

9. An expansion body connected to a distal portion of an outer tube and configured to expand in a radial direction, the expansion body comprising:
a plurality of main struts arranged at intervals in a circumferential direction and extending along an axis of the expansion body, each of the main struts includes a proximal side main strut, a proximal side sandwiching strut, a distal side sandwiching strut, and a distal side main strut;
each of the proximal side main struts being inclined so as to increase in a radial direction from a distal portion of the outer tube toward a distal direction, and the distal side main strut is inclined so as to increase in the radial direction from a force reception portion toward a proximal direction of the expansion body;
each of the proximal side sandwiching struts being inclined so as to decrease in the radial direction from a distal portion of the proximal side main strut toward the distal direction;
each of the distal side sandwiching struts being inclined so as to decrease in the radial direction from a proximal portion of the distal side main strut toward the proximal direction;
wherein the proximal side sandwiching strut and the distal side sandwiching strut are connected to each other by an inward protruding portion protruding inward in the radial direction; and
each of the plurality of main struts including an energy transfer element disposed at a position where one of the proximal side sandwiching strut and the distal side sandwiching strut of the main strut such that the energy transfer element sandwiches biological tissue with an other of the proximal side sandwiching strut and the distal side sandwiching strut of the main strut.

10. The expansion body according to claim 9, further comprising:
a plurality of sub-struts connected to the plurality of main struts, the plurality of sub-struts include a distal side sub-strut on a distal side of the inward protruding portion, and a proximal side sub-strut on a proximal side of the inward protruding portion.

11. The expansion body according to claim 10, further comprising:
a distal side support strut at a distal portion of the distal side sub-strut, the distal side support strut being connected to two circumferentially adjacent distal side main struts; and
a proximal side support strut at a proximal portion of the sub-strut, the proximal side support strut being connected to two circumferentially adjacent distal side sandwiching struts.

12. The expansion body according to claim 11, wherein the proximal side sub-strut is connected to two circumferentially adjacent proximal side main struts, the proximal side sub-strut including two inclined struts inclined with respect to an axis of the expansion body when viewed from a radially outer side.

13. The expansion body according to claim 12, wherein the two inclined struts extend toward the proximal direction while approaching the respective circumferentially adjacent proximal side main struts, and are connected to each other at a merging portion, and wherein the two inclined struts connected to the merging portion are plane-symmetrical with respect to a plane passing through the merging portion and the axis of the expansion body.

14. The expansion body according to claim 9, further comprising:
a pulling shaft configured to be disposed at least partially inside the outer tube, the pulling shaft being connected to the expansion body.

15. The expansion body according to claim 14, wherein the pulling shaft includes an inner tube movable inside the outer tube in an axial direction, and an engagement portion fixed to a distal portion of the inner tube, the engagement portion configured to be pulled toward a proximal direction by the inner tube to compress the expansion body in the axial direction.

16. A treatment method comprising:
performing maintenance treatment for maintaining a size of a through-hole formed in an atrial septum to allow a right atrium and a left atrium of a heart failure patient to communicate with each other with a medical device, the medical device including an elongated outer tube, an expansion body connected to a distal portion of the outer tube and configured to expand in a radial direction, a pulling shaft configured to slide with respect to the outer tube, the pulling shaft being disposed inside the outer tube, protruding from the distal portion of the outer tube, and being connected to a distal portion of the expansion body, a plurality of energy transfer elements disposed in the expansion body and configured to output energy, the expansion body including a plurality of main struts arranged at intervals in a circumferential direction and extending along an axis of the outer tube by a predetermined length, and a plurality of sub-struts connected to the plurality of main struts, at least one of the plurality of energy transfer elements is disposed on each of the plurality of main struts, each of the plurality of main struts includes a force reception portion configured to receive a pulling force from the pulling shaft, a portion of each of the plurality of main struts between the force reception portion and the energy transfer element is substantially parallel to an axis of the expansion body when viewed from a radially outer side, each of the plurality of sub-struts includes at least one support strut having two joint portions joined respectively to two circumferentially adjacent main struts among the plurality of main struts, and each of the plurality of support struts is formed to be longer than a linear distance between the two joint portions.

17. The method according to claim 16, further comprising:
applying energy to an edge portion of the through-hole through the energy transfer elements of the medical device to cauterize the edge portion of the through-hole.

18. The method according to claim 16, further comprising:
inserting a balloon catheter into a lumen of the pulling shaft from a proximal side of the pulling shaft;
disposing the balloon catheter inside the expansion body;
inflating a balloon of the balloon catheter by supplying an inflation fluid to the balloon; and
enlarging the through-hole with the balloon of the balloon catheter and the expansion body.

19. The method according to claim 18, further comprising:
discharging the inflation fluid from the balloon of the balloon catheter; and
confirming hemodynamics after the maintenance treatment.

20. The method according to claim 18, further comprising:
reducing a diameter of the expansion body; and
removing the expansion body from the through-hole.
